(19) United States
(12) Patent Application Publication
(10) Pub. No.: US 2011/0086923 A1
(43) Pub. Date: Apr. 14, 2011

(54) Method to increase oxygen in male and female sexual organs through the topical use of perfluorocarbons

(76) Inventors: Deborah P. Thompson, Durham, NC (US); Richard Kiral, Costa Mesa, CA (US); Chris Stern, Raleigh, NC (US)

(21) Appl. No.: 12/804,696

(22) Filed: Jul. 27, 2010

Related U.S. Application Data
(60) Provisional application No. 61/271,929, filed on Jul. 28, 2009.

(51) Int. Cl. A61K 31/025; A61K 31/02; A61P 15/00

(52) U.S. Cl. 514/747; 514/759

(57) ABSTRACT

The subject application provides for a method of increasing oxygen level in a sex organ of a subject comprising topically administering to the subject an amount of a perfluorocarbon effective to increase the oxygen level in the sex organ of the subject. The subject application also provides for the use of a perfluorocarbon in the manufacture of a composition for increasing oxygen level in a sex organ of a subject. The subject application also provides for a composition comprising a perfluorocarbon for use in increasing oxygen level in a sex organ of a subject.

This application claims the benefit of U.S. Provisional Application No. 61/271,929, filed Jul. 28, 2009, the entire content of which is hereby incorporated by reference herein.

Throughout this application various publications are referenced. Full citations for these references may be found at the end of the specification immediately preceding the claims. The disclosures of these publications in their entireties are hereby incorporated by reference into this application to more fully describe the state of the art to which this invention pertains.

### BACKGROUND OF THE INVENTION

Disorders of sexual function are common among men of all ages, ethnicities, and cultural backgrounds. It was estimated that more than 152 million men worldwide experienced erectile dysfunction (ED) in 1995, and that this number will rise by 170 million, to approximately 322 million by the year 2025. (Ayta, 1999)

Currently available erection-enhancement therapies include oral erection enhancement agents, local vasoactive agents administered via intracorporeal injections, intraurethral pessaries, or as topical cream (alprostadil cream Befar®, currently unavailable in the U.S.), constrictive ring and vacuum-induced tumescence, surgical corrections and penile prosthesis. These therapies are discussed in detail in Kandeel, F R et al. (2001) “Male Sexual Function and Its Disorders: Physiology, Pathophysiology, Clinical Investigation, and Treatment” Endocrine Review. 22(3):342-388.

Currently available oral erection enhancement agents include phosphodiesterase type 5 (PDE5) inhibitors sildenafil citrate (Viagra), tadalafil (Cialis) and vardenafil (Levitra). PDE5 inhibitors are the first FDA approved oral treatment available for ED. PDE5 inhibitors act by inhibiting cyclic guanosine monophosphate (cGMP) specific phosphodiesterase type 5 (PDE5), an enzyme that regulates blood flow in the penis. Specifically, PDE5 is an enzyme that accepts and breaks down cGMP. (Corbin, 2004; Daugan 2003)

Penile erection during sexual stimulation is caused by increased penile blood flow resulting from the relaxation of penile arteries and the smooth muscle of the corpus cavernosum (the erectile tissue). This response is mediated by the release of nitric oxide (NO) from nerve terminals and endothelial cells as a result of sexual stimulation. Nitric oxide activates the enzyme guanylate cyclase which stimulates the synthesis of cGMP in smooth muscle cells. cGMP relaxes smooth muscle and increases blood flow to the corpus cavernosum, resulting in an erection. (Burnett, 1997; Ignarro, 2002) PDE5 inhibitors inhibit the degradation of cGMP by phosphodiesterase type 5 (PDE5), increasing blood flow to the penis during sexual stimulation.

In addition to the therapies described above, a number of dietary supplements are sold and promoted for enhancement sexual performance. Many of such dietary supplements contain as active ingredients a blend of various components which purportedly have aphrodisiac properties and/or enhance sexual performance. For example, Sutramax™ is promoted as a sexual performance enhancement supplement for men. According to the manufacturer Natrient, LLC, Sutramax™ comprises Glycine Propionyl-L-Carnitine HCL (GlycoCarn®), which is promoted as a cardiovascular health and sports performance enhancement supplement claimed to increase nitric oxide (NO) retention, and various herbal ingredients including Butea Superba, Tribulus Terestis Extract, Pinus Radiata Extract, Grape Seed Extract, Eurycoma Longifolia Extract, Rhodiola Rosea Extract, Pomegranate Extract, Cocoa Bean Extract and Panax Ginseng Extract.

### SUMMARY OF THE INVENTION

Disclosed herein is a topical method for maintaining sexual function, i.e., maintaining overall sexual health, and enhancing sexual function. Specifically, disclosed herein is a method for increasing oxygen delivery to the sex organs of a subject, thereby increasing oxygen level and oxygen tension in the sex organ tissue for the maintenance and enhancement of male and female sexual function.

The subject application provides for a method of increasing oxygen level in a sex organ of a subject comprising topically administering to the subject an amount of a perfluorocarbon effective to increase the oxygen level in the sex organ of the subject.

The subject application also provides for the use of a perfluorocarbon in the manufacture of a composition for increasing oxygen level in a sex organ of a subject.

The subject application also provides for a composition comprising a perfluorocarbon for use in increasing oxygen level in a sex organ of a subject.

### DETAILED DESCRIPTION OF THE INVENTION

The subject application provides for a method of increasing oxygen level in a sex organ of a subject comprising topically administering to the subject an amount of a perfluorocarbon effective to increase the oxygen level in the sex organ of the subject.

In one embodiment, after the administration of the perfluorocarbon, oxygen tension in the sex organ of the subject is increased.

In one embodiment, the sex organ is the genitalia. In another embodiment, the perfluorocarbon is perfluoro(tert-butylcyclohexane).

In one embodiment, the perfluorocarbon is in the form of a gel. In another embodiment, the gel comprises 20-90 wt % perfluorocarbon. In another embodiment, the gel comprises 30-50 wt % perfluorocarbon. In another embodiment, the gel further comprises a surfactant.

In one embodiment, the gel comprises 1-5 wt % surfactants. In another embodiment, the surfactants include polyoxyethylene-polyoxypropylene block copolymers. In another embodiment, the polyoxyethylene-polyoxypropylene block copolymers include Poloxamer 105 and/or Poloxamer 188.

In one embodiment, the perfluorocarbon is administered with a warming agent. In another embodiment, the warming agent is capsaicin or cinnamon oil extract or any other warming agent.

In one embodiment, the perfluorocarbon is administered with a cooling agent. In another embodiment, the cooling agent is menthol.

In one embodiment, the perfluorocarbon is administered with a lubricant. In another embodiment, the lubricant is glycerol.

In one embodiment, the perfluorocarbon is administered periodically. In another embodiment, the perfluorocarbon is administered once daily. In yet another embodiment, the perfluorocarbon is administered twice daily.

In one embodiment, the subject is a mammal. In another embodiment, the mammal is a human. In another embodiment, the subject is male. In yet another embodiment, the subject is female.

The subject application also provides for the use of a perfluorocarbon in the manufacture of a composition for increasing oxygen level in a sex organ of a subject.

The subject application also provides for a composition comprising a perfluorocarbon for use in increasing oxygen level in a sex organ of a subject.

All combinations of the various elements described herein are within the scope of the invention.

### Terms

As used herein, and unless stated otherwise, each of the following terms shall have the definition set forth below.

“Biologically active agent” means a substance which has a beneficial or adverse effect on living matters.

“Effective” as in an amount effective to achieve an end means the quantity of a component that is sufficient to yield a desired response without undue adverse side effects (such as toxicity, irritation, or allergic response) commensurate with a reasonable benefit/risk ratio when used in the manner of this disclosure. For example, an amount effective to increase oxygen level in the sex organs of a subject without causing undue adverse side effects. The specific effective amount will vary with such factors as the particular condition being treated, the physical condition of the subject, the type of mammal being treated, the duration of the treatment, the nature of concurrent therapy (if any), and the specific formulations employed and the structure of the compounds or its derivatives.

“Gel” means a semi-solid or solid colloid (depending on concentration and/or temperature) of a solid/semi-solid and a liquid wherein a liquid dispersed phase is dispersed in a solid/semi-solid continuous medium. Some gels become fluids due to agitation then resume their gel structure when allowed to be undisturbed. Common pharmaceutical gels are solids which when applied and with motion allow the product to become temporarily a liquid phase so it applies smoothly, then becomes tacky then dries. Other gels are semi solid which are a semi-liquid, semi-solid mixture & when applied become tacky then dry.

“Oxygen tension” or “tissue oxygen tension” is the directly measured local partial pressure of oxygen in a specific tissue.

“Oxygenated perfluorocarbon” is a perfluorocarbon which is carrying oxygen at, for example, saturation or sub-saturation levels.

A “pharmaceutically acceptable carrier” as used herein refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject. The carrier may be liquid or solid and is selected with the planned manner of administration in mind.

A “salt” is salt of the instant compounds which have been modified by making acid or base salts of the compounds. The term “pharmaceutically acceptable salt” in this respect, refers to the relatively non-toxic, inorganic and organic acid or base addition salts of compounds of the present invention.

“Sex organ” or “sexual organ” means any of the anatomical parts of the body which are involved in sexual reproduction and constitute the reproductive system in a complex organism. In a preferred embodiment of this invention, the sex organ is the genitalia of the subject. As used herein, the “genitalia” refer to the externally visible sex organs: in males the penis, in females the clitoris and vulva.

“Surfactants” means wetting agents that lower the surface tension of a liquid, allowing easier spreading, and lower the interfacial tension between two liquids. In one embodiment of the present invention, the surfactant is polyethylene-polypropylene Glycol. In another embodiment of the present invention, the polyethylene-polypropylene Glycol (Generic Name Poloxamer) is Poloxamer 188 or Poloxamer 407. Poloxamer 188 can be obtained as Pluronic® F68. Poloxamer 407 can be obtained as Pluronic® F127. Pluronic® F68 and Pluronic® F127 can be obtained from, for example, Sigma-Aldrich Corp., St. Louis, Mo.

“Warming agent” as used herein is an agent which is capable of generating and transferring heat to the surface to which they are applied, i.e. the skin, and has a warming effect when applied to mammalian, e.g., human, skin. Similarly, a “cooling agent” as used herein is an agent which produce a cooling sensation when applied to mammalian, e.g., human, skin.

### Oxygen Tension And Mediation Of Vasoactive Substances

Studies have shown that oxygen tension plays an active role in regulating penile erection. Measurements of cavernosal blood pO₂ in human volunteer subjects indicate that oxygen tension changes rapidly from venous (˜35 mmHg) to arterial (˜100 mmHg) levels during the transition from the flaccid to the erect state. Maintenance of constant oxygen tension is a critical imperative in most tissues of the body but the penis is the only organ, which changes from venous to arterial oxygen tensions during the course of its normal function. This transition is the basis of a unique regulatory mechanism that takes advantage of key synthetic enzymes which utilize molecular oxygen as a co-substrate. NO synthase and prostaglandin synthase are two well-studied examples of a class of enzymes known as dioxygenases. At low oxygen tension, measured in the flaccid state of the penis, the synthesis of NO is inhibited, preventing travecular smooth muscle relaxation. This inhibition of NO production is probably necessary for the maintenance of penile flaccidity. Following vasodilation of the resistance arteries, the increase in arterial flow raises oxygen tension. In the oxygen enhanced environment, autonomic dilator nerves and the endothelium are able to synthesize NO, mediating trabecular smooth muscle relaxation. The synthesis of prostanoids is similarly regulated in the flaccid versus erect state. Therefore oxygen tension may regulate the types of vasoactive substances present in this vascular bed. At low oxygen tension, norepinephrine and endothelin-induced contraction may predominate, while at high oxygen tension, NO and prostalandins are produced due to the availability of molecular oxygen that is required for their synthesis. (Park 2009; Padmanabhan, 2007; Kim, 1993)

In addition, studies have also long used tissue oxygen tension as a measure of female sexual function. (Giuliano, 2001; Giuliano, 2002; Min, 2001)

### Perfluorocarbons

Perfluorocarbons (PFCs) possess the ability to dissolve large quantities of many gases at concentrations much larger than water, saline and plasma. In addition, PFCs can transport these gases to diffuse across distances. Thus, PFCs can be a convenient and inexpensive means to deliver high levels of oxygen or other therapeutic gases to tissues and organs.

PFCs that are commonly used in medical research are non-toxic, biologically inert, biostatic liquids at room temperature with densities of about 1.5-2.0 g/mL and high solubilities for oxygen and carbon dioxide. Such PFCs have been found to be efficient carriers of gases, both as emulsions for intravenous use and as neat liquids for liquid ventilation applications. Use of perfluorochemicals in biological gas exchange, for example as a blood substitute, or for intra-pulmonary or liquid ventilation applications, is described in U.S. Pat. No. 5,674,913, issued Oct. 7, 1997 to Clark, Jr., and in U.S. Pat. No. 5,840,767, issued Nov. 24, 1998 to Clark, Jr. et al., which are incorporated herein by reference.

Examples of PFCs that can be used according to the present invention include perfluorodecalin and perfluoro(tert-butylcyclohexane) (C₁₀F₂₀). Perfluoro(tert-butylcyclohexane) is available, for example, as Oxycyte™ from Oxygen Biotherapeutics Inc., Costa Mesa, Calif. In an embodiment, the perfluoro(tert-butylcyclohexane) has the following structure:

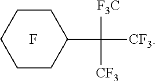

Oxycyte™ is a perfluorocarbon emulsion oxygen carrier. The active ingredient in Oxycyte™, perfluoro(tert-butylcyclohexane) (C₁₀F₂₀, MW-500), also known as F-tert-butylcyclohexane or “FtBu”, is a saturated alicyclic PFC. Perfluoro(tert-butylcyclohexane) is a colorless, completely inert, non-water soluble, non-lipophilic molecule, which is twice as dense as water, and boils at 147° C.

Oxycyte™ can be used as a PFC composition in the methods and uses described herein. Physical properties of F-tert-butylcyclohexane are as follows:

### Molecular Formula C₁₀F₂₀

Molecular Weight (g/mol) 500.08
Physical State @ Room Temp. Liquid
Density (g/mL) 1.97

### Boiling Point (° C.) 147

Vapor Pressure (mmHg) @ 25° C. 3.8
Vapor Pressure (mmHg) @ 37° C. 4.4

### Kinematic Viscosity (cP) 5.378

### Refractive Index @ 20° C. 1.3098

### Calculated Dipole Moment (Debye) 0.287

Calculated Surface Tension (dyne/cm) 14.4

The perfluorocarbon can be administered as a gel or an emulsion. The perfluorocarbon emulsions of the methods and uses of the invention include perfluorocarbon-in-water emulsions comprising a continuous aqueous phase and a discontinuous perfluorocarbon phase. The emulsions typically include emulsifiers, buffers, osmotic agents, and electrolytes. The perfluorocarbons are present in the emulsion from about 5% to 130% w/v. Embodiments include at least about 40%, 45%, 50%, 55%, 60%, 65%, 70%, 75%, 80% and 85% w/v. A 60% w/v perfluoro(tert-butylcyclohexane) emulsion may be used as the perfluorocarbon emulsion in one embodiment. Embodiments also include an egg yolk phospholipid emulsion buffered in an isotonic medium wherein the perfluorocarbon is present in the emulsion from about 5% to 130% w/v. Embodiments include at least about 40%, 45%, 50%, 55%, 60%, 65%, 70%, 75%, 80% and 85% w/v. A 60% w/v perfluoro(tert-butylcyclohexane) emulsion may be used as the perfluorocarbon emulsion in one embodiment of an egg yolk phospholipid emulsion buffered in an isotonic medium.

The perfluorocarbons employed in the methods described herein may be in compositions which may further comprise pharmaceutically acceptable carrier or cosmetic carrier and adjuvant(s) suitable for topical administration. Compositions suitable for topical administration are well known in the pharmaceutical and cosmetic arts. These compositions can be adapted to comprise oxygenated perfluorocarbon. The composition employed in the methods described herein may also comprise a pharmaceutically acceptable additive.

The multiplicity of configurations may contain additional beneficial active biological agents which further promote tissue health.

The perfluorocarbons may be in a salt form. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. These salts can be prepared in situ during the final isolation and purification of the compounds of the invention, or by separately reacting a purified compound of the invention in its free base or free acid form with a suitable organic or inorganic acid or base, and isolating the salt thus formed.

Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The compositions of this invention may be administered in forms detailed herein. The use of perfluorocarbon may be a component of a combination therapy or an adjunct therapy. The combination therapy can be sequential or simultaneous. The compounds can be administered independently by the same route or by two or more different routes of administration depending on the dosage forms employed. In an embodiment, a composition is provided comprising an amount of a perfluorocarbon effective to increase oxygen level in a sex organ of a subject as specified above and a pharmaceutical carrier.

The dosage of the compounds administered in treatment will vary depending upon factors such as the pharmacodynamic characteristics of a specific perfluorocarbon and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect. A dosage unit of the compounds may comprise a single compound or mixtures thereof with other compounds.

The compounds can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical or cosmetic practices. The compounds can be administered alone but are generally mixed with a pharmaceutically acceptable carrier. This carrier can be a solid or liquid, and the type of carrier is generally chosen based on the type of administration being used. Examples of suitable solid carriers include lactose, sucrose, gelatin and agar. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.). All of the aforementioned publications are incorporated by reference herein.

The PFC compositions may contain the any of the following non-toxic auxiliary substances:

The PFC compositions may contain antibacterial components which are non-injurious in use, for example, thimerosal, benzalkonium chloride, methyl and propyl paraben, benzyldodecinium bromide, benzyl alcohol, or phenylethanol.

The PFC compositions may also contain buffering ingredients such as sodium chloride, sodium acetate, gluconate buffers, phosphates, bicarbonate, citrate, borate, ACES, BES, BICINE, BIS-Tris, BIS-Tris Propane, HEPES, HEPPS, irnidazole, MES, MOPS, PIPES, TAPS, TES, and Tricine.

The PFC compositions may also contain a non-toxic pharmaceutical organic carrier, or with a non-toxic pharmaceutical inorganic carrier. Typical of pharmaceutically acceptable carriers are, for example, water, mixtures of water and water-miscible solvents such as lower alkanols or aralkanols, vegetable oils, peanut oil, polyalkylene glycols, petroleum based jelly, ethyl cellulose, ethyl oleate, carboxymethyl-cellulose, polyvinylpyrrolidone, isopropyl myristate and other conventionally employed acceptable carriers.

The PFC compositions may also contain non-toxic emulsifying, preserving, wetting agents, bodying agents, as for example, polyethylene glycols 200, 300, 400 and 600, carbowaxes 1,000, 1,500, 4,000, 6,000 and 10,000, antibacterial components such as quaternary ammonium compounds, phenylmercuric salts known to have cold sterilizing properties and which are non-injurious in use, thimerosal, methyl and propyl paraben, benzyl alcohol, phenyl ethanol, buffering ingredients such as sodium borate, sodium acetates, gluconate buffers, and other conventional ingredients such as sorbitan monolaurate, triethanolamine, oleate, polyoxyethylene sorbitan monopalmitylate, dioctyl sodium sulfosuccinate, monothioglycerol, thiosorbitol, ethylenediamine tetracetic.

The PFC compositions may also contain surfactants that might be employed include polysorbate surfactants, polyoxyethylene surfactants, phosphonates, saponins and polyethoxylated castor oils, but preferably the polyethoxylated castor oils. These surfactants are commercially available. The polyethoxylated castor oils are sold, for example, by BASF under the trademark Cremaphor.

The PFC compositions may also contain wetting agents commonly used in ophthalmic solutions such as carboxymethylcellulose, hydroxypropyl methylcellulose, glycerin, mannitol, polyvinyl alcohol or hydroxyethylcellulose and the diluting agent may be water, distilled water, sterile water, or artificial tears, wherein the wetting agent is present in an amount of about 0.001% to about 10%.

The formulation of this invention may be varied to include acids and bases to adjust the pH; tonicity imparting agents such as sorbitol, glycerin and dextrose; other viscosity imparting agents such as sodium carboxymethylcellulose, microcrystalline cellulose, polyvinylpyrrolidone, polyvinyl alcohol and other gums; suitable absorption enhancers, such as surfactants, bile acids; stabilizing agents such as antioxidants, like bisulfites and ascorbates; metal chelating agents, such as sodium edetate; and drug solubility enhancers, such as polyethylene glycols. These additional ingredients help make commercial solutions with adequate stability so that they need not be compounded on demand.

Other materials as well as processing techniques and the like are set forth in Part 8 of Remington's Pharmaceutical Sciences, 17th edition, 1985, Mack Publishing Company, Easton, Pa., and International Programme on Chemical Safety (IPCS), which is incorporated herein by reference.

It is understood that where a parameter range is provided, all integers within that range, and tenths thereof, are also provided by the invention. For example, “30-50%” includes 30.0%, 30.1%, 30.2%, 30.3%, 30.4% etc. up to 50.0%.

All combinations of the various elements are within the scope of the invention.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

Disclosed herein are methods using perfluorocarbon compositions for increasing oxygen level in tissue.

### Example 1

Oxycyte™ emulsion (60% wt/vol. PFC) was tested systemically via intravenous administration in Sprauge Dawley rats, Cynomolgus Monkeys and humans.

The Oxycyte™ emulsion was found to be well tolerated and had no toxicity.

### Example 2

A perfluorocarbon is administered topically to sex organs of a human male subject. Local oxygen tension and nocturnal erections are evaluated. Changes in Quality of life (QOL) data is also collected and assessed.

Oxygen level and oxygen tension in the tissue is found to have increased. In addition, Quality of life of the subject is found to have improved. Moreover, the perfluorocarbon is found to be well tolerated and had no toxicity.

### Example 3

A gel composition comprising Oxycyte™ is topically administered to sex organs of male and female human subjects. The Oxycyte™ gel is administered once or twice daily. Local oxygen tension and nocturnal erections (in males) are evaluated. Changes in Quality of life (QOL) data is also collected and assessed.

Oxygen level and oxygen tension in the tissue is found to have increased. In addition, Quality of life of the subject is found to have improved. Moreover, the perfluorocarbon composition is found to be well tolerated and had no toxicity.

### REFERENCES

1. Ayta I A, KcKinlay J B, Krane R J (1999) “The likely worldwide increase in erectile dysfunction between 1995 and 2025 and some possible policy consequences” Br J Urol Int, 84:50-56.

2. Burnett, A L. (January 1997) “Nitric oxide in the penis: physiology and pathology” The Journal of Urology, 157(1):320-324.

3. Corbin, J D (2004) “Mechanisms of action of PDE5 inhibition in erectile dysfunction” International Journal of Impotence Research, 16:S4-S7.

4. Daugan, A. et al. (Oct. 9, 2003). “The discovery of tadalafil: a novel and highly selective PDE5 inhibitor. 1: 5,6,11,11a-tetrahydro-1H-imidazo[1′,5′:1,6]pyrido[3,4-b]indole-1,3(2H)-dione analogues” Journal of Medicinal Chemistry, 46(21):4525-32. PMID 14521414.

5. Giuliano, F. et al. (2001) “Vaginal physiological changes in a model of sexual arousal in anesthetized rats” Am J Physiol Regul Integr Comp Physiol, 281: R140-R149.

6. Giuliano, F. et al. (2002) “Neurophysiology and Pharmacology of Female Genital Sexual Response” Journal of Sex & Marital Therapy, 1521-0715, Volume 28, Issue 1, Supplement 1, Pgs 101-121.

7. Ignarro, L J (2002) “Nitric Oxide As a Unique Signaling Molecule In The vascular system: A Historical Overview” Journal of Physiology and Pharmacology, 53(4):503-514.

8. Kandeel, F R et al. (2001) “Male Sexual Function and Its Disorders: Physiology, Pathophysiology, Clinical Investigation, and Treatment” Endocrine Review, 22(3):342-388.

9. Kim, N., et al. (1993) “Oxygen Tension Regulates the Nitric Oxide Pathway” J. Clin. Invest, 91:437-442.

10. Min, K. et al. (2001) “Hemodynamic Evaluation of the Female Sexual Arousal Response in an Animal Model” Journal of Sex & Marital Therapy, 1521-0715, 27(5):557-565.

11. Padmanabhan P and McCullough A R (March/April 2007) “Penile Oxygen Saturation in the Flaccid and Erect Penis in Men with and Without Erectile Dysfuntion” Journal of Andrology, 28(2):223-228.

12. Park, J K. Et al. “The Role of Oxygen Tension in Penile Erection and Its Relationship to Erectile Dysfunction” Digital Urology Journal, (http://www.duj.com/Article/Nehra/Nehra.html) retrieved Jul. 22, 2009.

13. Sommer, F., et al. (April 2001) “Measurement Of Vaginal And Minor Labial Oxygen Tension For The Evaluation Of Female Sexual Function” The Journal of Urology, 165(4):1181-1184.

1. A method of increasing oxygen level in a sex organ of a subject comprising topically administering to the subject an amount of a perfluorocarbon effective to increase the oxygen level in the sex organ of the subject.

2. The method of claim 1, wherein after the administration of the perfluorocarbon, oxygen tension in the sex organ of the subject is increased.

3. The method of claim 1, wherein the sex organ is the genitalia.

4. The method of claim 1, wherein the perfluorocarbon is perfluoro(tert-butylcyclohexane).

5. The method of claim 1, wherein the perfluorocarbon is in the form of a gel.

6. The method of claim 5, wherein the gel comprises 20-90 wt % perfluorocarbon.

7. The method of claim 6, wherein the gel comprises 30-50 wt % perfluorocarbon.

8. The method of claim 5, wherein the gel further comprises a surfactant.

9. The method of any claim 1, wherein the perfluorocarbon is administered with a warming agent.

10. The method of claim 9, wherein the warming agent is capsaicin or cinnamon oil extract.

11. The method of claim 1, wherein the perfluorocarbon is administered with a cooling agent.

12. The method of claim 11, wherein the warming agent is menthol.

13. The method of claim 1, wherein the perfluorocarbon is administered with a lubricant.

14. The method of claim 13, wherein the lubricant is glycerol.

15. The method of claim 1, wherein the perfluorocarbon is administered periodically.

16. The method of claim 15, wherein the perfluorocarbon is administered once daily.

17. The method of claim 15, wherein the perfluorocarbon is administered twice daily.

18. The method of claim 1, wherein the subject is a mammal.

19. The method of claim 18, wherein the mammal is a human.

20. The method of claim 1, where in the subject is male.

21-22. (canceled)
